(19) United States
(12) Patent Application Publication
(10) Pub. No.: US 2024/0130959 A1
(43) Pub. Date: Apr. 25, 2024

(54) CREAM FOR TREATING ACNE AND MELASMA

(71) Applicant: Maria TRIPOD, Bonne (FR)

(72) Inventor: Maria TRIPOD, Bonne (FR)

(21) Appl. No.: 17/769,493

(22) PCT Filed: Sep. 23, 2020
(86) PCT No.: PCT/IB2020/058912

§ 371 Date: Apr. 14, 2022

(30) Foreign Application Priority Data
Oct. 15, 2019 (PT) 115850 S

(51) Int. Cl. A61K 8/9771; A61K 8/19; A61K 8/34; A61K 8/49; A61K 8/60; A61K 8/67; A61K 8/73; A61K 8/9789; A61K 31/047; A61K 31/351; A61K 31/375; A61K 31/4174; A61K 31/455; A61K 31/7034; A61K 31/728; A61K 33/10; A61K 36/16; A61K 36/258; A61K 36/87; A61Q 19/02

(52) U.S. Cl. CPC A61K 8/9771; A61K 8/19; A61K 8/345; A61K 8/4946; A61K 8/498; A61K 8/602; A61K 8/675; A61K 8/676; A61K 8/735; A61K 8/9789; A61K 31/047; A61K 31/351; A61K 31/375; A61K 31/4174; A61K 31/455; A61K 31/7034; A61K 31/728; A61K 33/10; A61K 36/16; A61K 36/258; A61K 36/87; A61Q 19/02

(57) ABSTRACT

The product is made of phytotherapeutic active substances extracted from roots, stems and leaves of medicinal plants, biological emulsifier, synthetic active substances of organic origin in small quantities, minerals and water. The product is made in the oil, water and dry phases according to best manufacturing practice to obtain a high-quality cream to treat acne and melasma safely, quickly and effectively. The cream can be used to treat all skin types, is applied to the affected area, and has anti-inflammatory, antiseptic, antioxidant, healing, anti-fungicidal, moisturizing and skin-lightening properties.

### SUMMARY OF THE INVENTION

The product is made with herbal active ingredients extracted from roots, stems and leaves of medicinal plants, biological emulsifier, synthetic actives of organic origin in small amounts, minerals and water.

The product is made in the oily, aqueous and dry phases following the recommendations of Good Manufacturing Practices with. The objective of obtaining a good quality cream to fight acne and melasma in a safe, fast and effective way.

The cream is for the treatment of all skin types, to be applied to the injured or affected region and has anti-inflammatory, antiseptic, antioxidant, healing, antifungal, moisturizing and skin whitening properties.

### TECHNICAL AREA OF THE INVENTION

The skin is the largest organ in the human body, it is responsible for protection, temperature regulation and sensitivity of the body. To keep the skin within healthy and aesthetic standards, it is necessary to correct the imperfections and stains caused by acne and or melasma. For this treatment, the product proposed here, prepared with specific active principles, obtains the desired result and meets the patient's expectations.

The product is prepared with synthetic active principles of organic origin in small amounts, water, biological emulsifier and largely by phytotherapic actives.

The herbal active ingredients used in the formula are products of plant origin and their benefits are proven by the WHO (World Health Organization) through pharmacological studies. These actives have specific properties such as healing, antiseptic, anti-inflammatory, antioxidant, moisturizing and skin lightening properties.

The purpose of the formula is to combat fungi that lodge in the skin, by means of a substance that has the property of fungicide, to eliminate or reduce the excess pigmentation that causes melasma and to fight inflammation.

Acnes are skin inflammations that, depending on the degree of injury, can leave scars and spots in the affected region caused by factors such as:
a. Hormonal
b. Stress
c. General patient hygiene
d. Old injuries
e. Exposure to the sun without protection
f. Skin moisture
g. Skin oiliness
h. Food
i. Side effect of some medications
j. Others

Melasmas are spots on the skin due to an excessive increase in melanin in the body caused by factors such as:
a. Hormonal
b. Stress
c. Chemical composition of the skin
d. General patient hygiene
e. Old skin lesions
f. Unprotected sun exposure
g. skin moisture
h. Food
i. Contraceptives
j. Others

### ACTIVE INGREDIENTS AND THEIR PROPERTIES

Polawax: Emulsifier and stabilizer. It is a wax of natural origin that keeps the product hydrated, giving it shine and consistency in order to provide adequate viscosity to facilitate the application of the cream and aid in the absorption of active ingredients by the skin.

Ginko biloba: Anti-inflammatory and antioxidant. It is a medicinal plant very rich in flavonoids and terpenoids.

Nettle or nettle: anti-inflammatory and helps to reduce skin oiliness, preventing the appearance of acne. It is a medicinal plant rich in vitamins and iron.

Gayuba or bearberry: antiseptic and anti-inflammatory. For topical use on wounds and dermal ulcerations. Contains glycosides, flavonoids and arbutin.

Garnet or pomegranate: Antiseptic. It is a fruit rich in minerals such as phosphorus, sodium, calcium and iron.

Ginseng: Anti-inflammatory, antioxidant and immune system stimulant. Ginseng contains vitamins B1, B2 and B3, cobalt iron, polysaccharides, flavonoids, essential oils, magnesium, manganese, copper and calcium. Magnesium works in the recovery of damaged cells and helps fight acne. Manganese acts against free radicals responsible for premature skin aging. Copper helps in the absorption of iron helping in the good healing of wounds maintaining the elasticity of the skin. Calcium stimulates collagen and is important for maintaining firm, healthy skin.

Grape: antioxidant. It is rich in resveratrol and phenolic compounds that maintain cell development.

Kojic Acid: antiseptic, antioxidant. It inhibits the formation of melanin, combating hyperpigmentation, reducing or eliminating blemishes, whitening the skin and preventing the appearance of new ones.

Vitamin C: antioxidant, anti-aging, immunoprotective and anti-inflammatory, combats the action of free radicals, stimulates collagen formation, protects the skin from the effects of the sun and improves its elasticity.

Hyaluronic acid: moisturizes the skin. It helps in the production of collagen, prevents and controls sagging giving volume, firmness and elasticity to the skin. Helps with product viscosity.

Dolomite: alkaline, anti-inflammatory, analgesic, remineralizing, decongestant, toning and relaxing

Clotrimazole: broad spectrum fungicide, very effective in combating a wide range of fungi.

Alpha arbutin: works to combat hyper-pigmentation, eliminates or reduces skin blemishes.

Caprylyl: moisturizing and bactericidal preservative. Helps keep the skin hydrated.

Vitamin B3: anti-inflammatory and moisturizing. Prevents the appearance of blemishes and irritations, especially on sensitive skin. It acts to control the action of the sebaceous glands, preventing the appearance of acne.

Palm kernel: the oil is composed of lauric acid and myristic acid. Lauric acid has anti-inflammatory, antibacterial and antifungal properties and myristic acid aids in the absorption of active ingredients by the skin.

EDTA: chelating agent. It gives stability to the product, preventing changes in color, smell and appearance.

1. I file a patent application.

2. It is a product invention with the aim of fighting acne and melasma, regenerating the skin's health.

3. Below is the product formulation and procedures.

4. Preparation follows five phases:

5. The association of several actives, melasma inhibitor, fungicide, antioxidant, immuno-protective anti-inflammatory, moisturizing and antiseptic. It has a very positive result in the treatment of acne with closed comedones or open “white spots”, at the same time it lightens the marks on the skin caused by inflammation caused by acne, hydrates the skin giving it a pleasant and healthy texture. With an anti-allergenic test demonstrating that the association of substances proposed here does not have a negative impact on the skin.

6. Phase 1: oily

7. The oily base, placing the Polawax and the palm kernel to melt over medium heat.
Mix water with EDTA
Add the water mixed with EDTA to the oil base in a water bath, stirring constantly

| | | |
|---|---|---|
| Polawax | 8.0 | g |
| Palm kernel | 2.0 | g |
| EDTA | 0.5 | g |
| Water | 20.0 | g |

Phase 2: Active extracts (liquid)
Mix all the ingredients:

| | |
|---|---|
| Ginkgo biloba | 3.0 ml |
| Ginseng | 3.0 ml |
| Grape | 4.0 ml |
| Caprylyl | 0.5 ml |

Phase 3: Dry
Mix all the ingredients:

| | |
|---|---|
| Kojic Acid | 1.0 g |
| vitamin B3 | 1.0 g |
| Alpha arbutin | 1.0 g |
| Vitamin C | 2.0 g |
| Dolomite | 6.0 g |
| Hyaluronic acid | 1.0 g |
| Clotrimazole | 1.0 g |

Phase 4:
Mix phase 2 with phase 3
Phase 5:
Mix phase 4 with phase 1
